(19) United States
(12) Patent Application Publication
(10) Pub. No.: US 2011/0054627 A1
(43) Pub. Date: Mar. 3, 2011

(54) Biologic Soft Tissue Arthroplasty Spacer and Joint Resurfacing of Wrist and Hand

(76) Inventor: Brian J. Bear, Rockford, IL (US)

(21) Appl. No.: 12/870,969

(22) Filed: Aug. 30, 2010

Related U.S. Application Data
(60) Provisional application No. 61/238,745, filed on Sep. 1, 2009.

(51) Int. Cl. A61F 2/42

(52) U.S. Cl. 623/21.12

(57) ABSTRACT

A arthroplasty spacer formed of a biologic soft tissue and methods for surgically treating symptomatic trapezio metacarpal joint arthritis and symptomatic arthritis of scapho trapezial trapezoid joint using the biologic soft tissue spacer are provided.

CROSS-REFERENCE TO RELATED PATENT APPLICATION

This patent application claims the benefit of U.S. Provisional Patent Application No. 61/238,745, filed Sep. 1, 2009, the entire teachings and disclosure of which are incorporated herein by reference thereto.

### FIELD OF THE INVENTION

This invention generally relates to implants for surgical treatment of joints, particularly implants for surgical treatment of symptomatic trapezio metacarpal joint arthritis and symptomatic arthritis of the scapho trapezial trapezoid (STT) joint.

### BACKGROUND OF THE INVENTION

Trapeziometacarpal arthritis is common condition affecting a large number of adults. Trapezium excision with and without anterior oblique ligament reconstruction/soft tissue interposition are common surgical treatment options for symptomatic trapezio metacarpal arthritis. Proximal migration of the thumb metacarpal with subsequent first metacarpal scaphoid impingement, clinically significant scapho trapezoid arthritis, and first/second metacarpal impingement are known causes of persistent pain after primary and revision trapeziometacarpal joint arthroplasty where the entire trapezium or a portion of the trapezium is resected. Loss of thumb length with and without first metacarpal scaphoid impingement is thought to be a cause of pain and weakness in failed trapezium excision surgery. There are currently no known biologic commercially available implants designed exclusively for treatment of primary or revision trapeziometacarpal arthritis with or without scapho trapezial trapezoid arthritis.

Arthritis of the scapho trapezial trapezoid (STT) joint is a known condition that can cause significant wrist and hand pain. Current treatment options for STT arthritis include STT joint fusion, partial or complete trapezial resection with partial trapezoid resection, partial or complete trapezial resection with partial trapezoid resection with autograft soft tissue interposition, distal pole scaphoid excision with and without soft tissue interposition. There is currently no known consensus on an optimal surgical treatment modality. STT fusion is technically difficult and significantly reduces wrist motion. There is significant morbidity concerns and complications associated with STT fusion in published peer reviewed medical journals. Trapezial resection with partial trapezoid resection is indicated if concomitant symptomatic trapeziometacarpal arthritis is present. It is not indicated for isolated symptomatic STT arthritis without trapeziometacarpal arthritis. Distal pole scaphoid excision with and without allograft interposition is an option for symptomatic STT arthritis. There are concerns that volar flexion of the scaphoid may occur with distal pole scaphoid excision and that this may be a risk factor for carpal instability and arthritis and secondary to carpal instability. There are currently no known biologic or synthetic commercially available implants designed for the treatment of isolated STT arthritis.

The invention provides novel implants and methods for surgical treatment of symptomatic trapezio metacarpal joint arthritis and isolated STT arthritis. These and other advantages of the invention, as well as additional inventive features, will be apparent from the description of the invention provided herein.

### BRIEF SUMMARY OF THE INVENTION

One inventive aspect is direct toward an arthroplasty spacer. The arthroplasty spacer is formed of a biologic soft tissue and includes a body configured to fit in an arthroplasty space.

According to one embodiment, the arthroplasty spacer has the body, which is configured to replace a resected trapezium for a trapezio metacarpal joint arthroplasty. In this embodiment the body is configured to be slightly larger than the resected trapezium. The body can have a rectangular box like shape and can also include a radial extension extending generally vertically upward from a first side of the body. The body is placed in the arthroplasty space created from the resected trapezium, and the radial extension is placed adjacent a thumb radial metacarpal base to prevent lateral subluxation of the thumb metacarpal.

According to another embodiment, the arthroplasty spacer can further include a scaphotrapezoid extension extending generally horizontally from a second side of the body, the first side and the second side being on opposite ends of the body, such that the radial extension, the body, and the scaphotrapezoid extension form a stair-like shape. In this embodiment, the scaphotrapezoid extension is configured to cover an approximate area of a scapho trapezoid joint and interposed into the scapho trapezoid joint in an arthroplasty space created from a partial resection of a trapezoid to resurface an arthritic scapho trapezoid joint.

According to yet another embodiment, the arthroplasty spacer can also include a first/second metacarpal extension extending generally vertically upward from the second side of the body, such that the first/second metacarpal extension and the second side of the body and the scaphotrapezoid extension generally form L-like shape, and the radial extension and a third side of the body and the first/second metacarpal extension generally form a U-like shape. The first/second metacarpal extension is place between first and second metacarpals to provide a soft tissue interposition.

According to another embodiment, the body of the arthroplasty spacer is configured to fit the arthroplasty space created from a partial resection of a distal scaphoid to resurface an arthritic scapho trapezial trapezoid (STT) joint.

The arthroplasty spacer according to various embodiments of the present invention can be formed of human allograft, autograft or xenograft tissues, which can be selected from a group consisting of tendon, dermal, fibro cartilage, labral, meniscal tissues and a mixture thereof. Further, the body of the arthroplasty spacer can be a compact block of biologic soft tissue formed by folding or manipulating the biologic soft tissue onto itself or sectioning, which is sewn together using a suture.

Another inventive aspect is directed toward a method for surgically treating symptomatic trapezio metacarpal joint arthritis. The method includes steps of removing a trapezium; preparing an arthroplasty space created from the removed trapezium, such that the arthroplasty space is mobile and free of scar tissue; measuring a size of the arthroplasty space; selecting a biologic soft tissue spacer according to the size of the arthroplasty space; placing the biologic soft tissue spacer in the arthroplasty space; and securing the biologic soft tissue spacer to a scaphoid. In some embodiments, the biologic soft tissue spacer is formed of human allograft, autograft or xenograft tissues and configured to replace the trapezium. In such embodiments, the biologic soft tissue spacer includes a body, which is configured to be slightly larger than the trapezium.

According to one embodiment, the biologic soft tissue spacer further includes a radial extension. In such embodiment, placing the biologic soft tissue spacer includes arranging the body in the arthroplasty space created from the removed trapezium, and placing the radial extension adjacent a thumb radial metacarpal base to prevent lateral subluxation of the thumb metacarpal. The method can further include securing the radial extension to the thumb radial metacarpal base by suturing to local soft tissue at the thumb radial metacarpal base or via a bone tunnel or a suture anchor placed in the thumb radial metacarpal base.

According another embodiment, the method further includes a step of resecting about 2-3 mm of a proximal trapezoid, and preparing a scapho trapezoid arthroplasty space created therein. In this embodiment the biologic soft tissue spacer is selected to include a scaphotrapezoid extension extending from the body. The step of placing the biologic soft tissue spacer includes arranging the body in the arthroplasty space created from the removed trapezium and placing the scaphotrapezoid extension in the scapho trapezoid arthroplasty space to also treat a symptomatic scapho trapezial arthritis.

According to yet another embodiment, the biologic soft tissue spacer further includes a first/second metacarpal extension. In this embodiment, the step of placing the biologic soft tissue spacer includes arranging the body in the arthroplasty space created from the removed trapezium and placing the first/second metacarpal extension between a first metacarpal and a second metacarpal to provide a soft tissue interposition to treat a symptomatic first/second metacarpal arthritis or impingement.

In one embodiment, securing the biologic soft tissue spacer includes placing a suture anchor into around the center of a distal pole of the scaphoid, securing a suture having limbs to the suture anchor, passing the limbs through the biologic soft tissue spacer, and tying the limbs together on a radial side of the biologic soft tissue spacer, such that the biologic soft tissue spacer is secured to the scaphoid.

When a volar approach is used, the method further includes a step of repairing a thenar musculature and a remaining capsule over the biologic soft tissue spacer. When a dorsal approach is used, the method includes a step of repairing capsule over the biologic tissue spacer using non-absorbable sutures.

Yet another inventive aspect is directed toward a method for surgically treating symptomatic arthritis of scapho trapezial trapezoid (STT) joint. The method includes steps of resecting about 2-3 mm of a distal scaphoid to create an arthroplasty space; measuring a surface area of the distal scaphoid in the STT joint; selecting a biologic soft tissue spacer according to the surface area of the distal scaphoid; placing the biologic soft tissue spacer in the arthroplasty space; and securing the biologic soft tissue spacer to the distal scaphoid.

According to one embodiment, the step of placing the biologic soft tissue spacer includes deviating a wrist ulnarly and setting the biologic soft tissue spacer in the STT joint such that the biologic soft tissue spacer generally covers the entire distal scaphoid. The step of securing the biologic soft tissue spacer includes placing a suture anchor into the distal scaphoid before setting the biologic soft tissue spacer in the STT joint, and passing limbs of a suture anchor suture through the biologic soft tissue spacer, and tying the limbs together on a radial side of the biologic soft tissue spacer, such that the biologic soft tissue spacer is secured to the scaphoid.

The method can further include steps of placing a non-absorbable suture through the dorsal half of the biologic soft tissue spacer in a mattress fashion, and passing the suture through a dorsal STT joint capsule and through a overlaying extensor carpi radialis longus tendon. The method can also include steps of confirming that a scapholunate angle is 60° or less using a flouroscopic imaging, and placing a smooth 0.045″, 0.055″ or 0.065″ K-wire through a distal third of the scapohid in a radial to ulnar direction and driving the K-wire into a capitate using a power drill to stabilize the scaphoid. Then the suture is tied to stabilize a distal pole of the scaphoid to the dorsal capsule and the overlaying extensor carpi radialis brevis tendon to prevent volar flexion of the scaphoid.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 is a perspective view of an implant according to a first embodiment of the present invention;

FIG. 2 is a perspective view of an implant according to a second embodiment of the present invention;

FIG. 3 is a perspective view of an implant according to a third embodiment of the present invention;

FIG. 4 is a schematic view of the implant of FIG. 1 used in a trapezio metacarpal joint arthroplasty procedure according to an embodiment of the present invention;

FIG. 5 is a schematic view of the implant of FIG. 1 without a radial extension used in a trapezio metacarpal joint arthroplasty procedure according to an embodiment of the present invention;

FIG. 6 is a schematic view of the implant of FIG. 2 used in a trapezio metacarpal joint arthroplasty procedure according to an embodiment of the present invention;

FIG. 7 is a schematic view of the implant of FIG. 3 used in a trapezio metacarpal joint arthroplasty procedure according to an embodiment of the present invention;

FIG. 8 is a schematic view a scapho trapezial trapezoid (STT) joint showing a resection area for an implant procedure according to an embodiment of the present invention;

FIG. 9 is a schematic view of a STT implant used in an implant procedure according to an embodiment of the present invention; and

FIG. 10 is a perspective view of the STT implant of FIG. 9.

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

### Implants for Surgical Treatment of Symptomatic Trapezio Metacarpal Joint Arthritis

Implants according to various embodiments of the present invention are designed to be simple to use, effective and time efficient. It obviates the need for autograft graft harvesting therefore reducing anesthesia and operating room time. It is more durable than auto graft tendon and will create a more durable resurfacing and arthroplasty spacer. The material properties of the implant are such that it is easy to use and stable after suture fixation. Traditional tendon interposition arthroplasty can be difficult to reliably secure to bone and maintain position in the arthroplasty space without extrusion with loading of the joint.

The implants according to embodiments of the present invention are designed to be utilized in primary and/or revision trapezio metacarpal joint arthroplasty procedures. It is designed as a xenograft , allograft or autograft soft tissue spacer to maintain first carpometacarpal joint arthroplasty space and overall thumb length status post trapezium excision for first carpometacarpal joint arthritis. The implants can also be used in treatment of symptomatic trapeziometacarpal arthritis with concomitant symptomatic scaphotrapezoid arthritis, andin treatment of failed first carpometacarpal arthroplasty surgery when first metacarpal scaphoid impingement or first metacarpal second metacarpal impingement occurs. Further, the implants can be used in revision and/or primary trapezio metacarpal joint arthroplasty procedures in the treatment of symptomatic arthritis of the trapezio metacarpal joint with concomitant symptomatic scaphotrapezoid arthritis. It also can be utilized in younger higher demand patients with symptomatic arthritis of the trapezio metacarpal joint with or without symptomatic scaphotrapezoid arthritis where preservation of thumb length and first carpo metacarpal arthroplasty space is thought to be important.

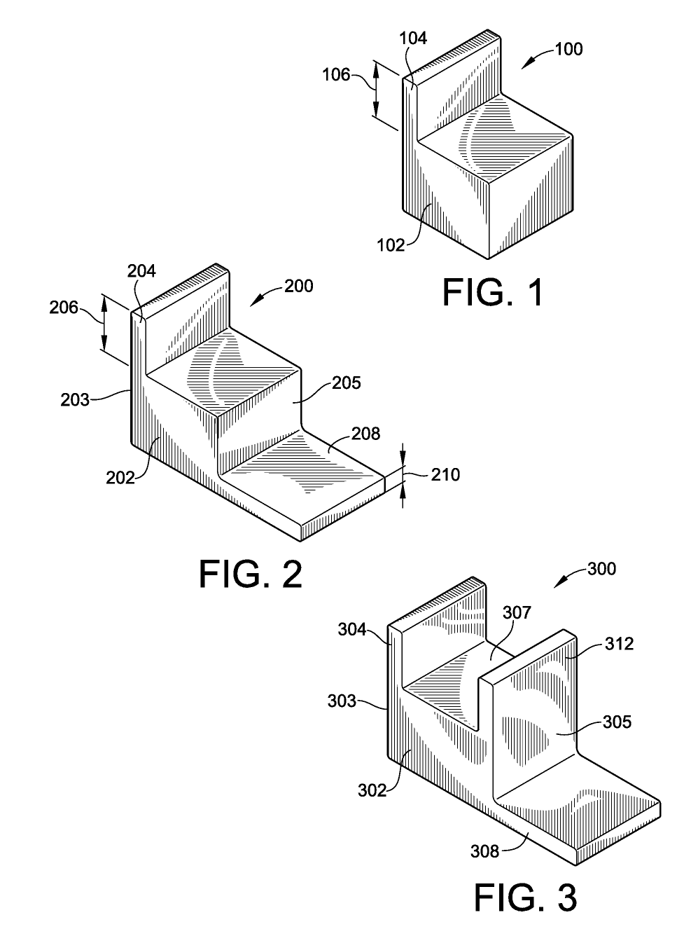

FIGS. 1-3 show Trapeziometacarpal Biologic Soft Tissue Arthroplasty Spacers according to embodiments of the present invention. The Trapeziometacarpal Biologic Soft Tissue Arthroplasty Spacer is also referred to herein as a spacer, an implant, or other like terms. The implants according to embodiments of the present invention are made from human allograft, autograft or xenograft tissues including but not limited to tendon, dermal, fibro cartilage, labral or meniscal tissues.

FIG. 1 shows a standard implant for an isolated trapezoid metacarpal arthritis treatment according to first embodiment of the present invention. As shown, the standard implant 100 has a body 102 having a rectangular box like shape and a radial extension 104 that extend vertically upward from the body, such that the implant 100 forms a chair like shape. The radial extension 104 can be secured to the thumb radial metacarpal base to prevent lateral subluxation of the metacarpal base. The body 102 is configured to be slightly larger than the approximate size of the trapezium that has been resected. In one embodiment, the radial extension 104 has a height 106 of between about 0.5 cm-2 cm, preferably between about 0.7 cm-1.5 cm, more preferably about 1 cm. The standard implant 100 can be used in treatment of primary symptomatic arthritis of the trapezio metacarpal joint, primary symptomatic arthritis of the trapeziometacarpal joint with co-existing arthritis of the scapho trapezial joint. Although, the implant 100 is shown with the radial extension 104, the radial extension 104 is optional. Thus, in other embodiments, the implant 100 may only include the body 102 without the radial extension 104.

FIG. 2 shows an implant according to second embodiment of the present invention. The implant 200 is similarly configured as the standard implant 100 of FIG. 1, and includes a body 202 and a radial extension 204. Additionally, the implant 200 includes a scapho trapezoid extension 208. As shown, the scapho trapezoid extension 208 is an proximal ulnar based extension that projects off the body 202 that is designed to be interposed into the scapho trapezoid joint when symptomatic scapho trapezoid arthritis is present with trapeziometacarpal and scapho trapezial arthritis. This is designed to resurface an arthritic scapho trapezoid joint after partially resecting the base of the trapezoid. As it was with the standard implant 100, the body 202 of the implant 200 has a rectangular box like shape including six sides and is configured to be slightly larger than the approximate size of the resected trapezium. In this embodiment, the radial extension 204 extends generally vertically upward from a first side 203 of the body 202, and the scaphotrapezoid extension 208 extends generally horizontally from a second side 205 of the body 202, such that the radial extension 204, the body 202, and the scaphotrapezoid extension 208 form a stair-like shape. The scapho trapezoid extension 208 is configured to cover the approximate area of the scapho trapezoid joint. In one embodiment the radial extension 124 has a height 206 of between about 0.5 cm-2 cm, preferably between about 0.7 cm-1.5 cm, more preferably about 1 cm; and the scapho trapezoid extension 208 has a height of between about 1 mm-3 mm, preferably between about 1.5 mm-2.5 mm, and more preferably about 2 mm.

FIG. 3 shows a revision implant according to third embodiment of the present invention. The revision implant 300 is similarly configured as the implant 200, and includes a body 302, a radial extension 304 and a scapho trapezoid extension 308. Additionally, the revision implant 300 includes a first/second metacarpal extension 312. As shown, the radial extension 304 extends generally vertically upward from a first side 303 of the rectangular box like shaped body 302, the scaphotrapezoid extension 308 extends generally horizontally form a second side 305 of the body 302, and the first/second metacarpal extension 312 extends generally vertically upward from the second side 305 of the body 302. As such, the first/second metacarpal extension 312, the second side 305, and the scaphotrapezoid extension 308 generally from a L-like shape, while the radial extension 304, a third side 307 of the body 302, and the first/second metacarpal extension 312 generally forma U-like shape. The first/second metacarpal extension 312 is a distal ulnar based extension that is designed to provide a soft tissue interposition when symptomatic first/second metacarpal arthritis and/or impingement is thought to be causing pain in failed previous trapeziometacarpal surgery with or without symptomatic first metacarpal scaphoid impingement, scaphotrapezoid arthritis, scapho trapezial arthritis.

There are right and left implants for each design. The implants can be configured according to the size of the trapeziometacarpal joint that is being reconstructed. For example, it is contemplated that up to three standard sizes of each design can be made available to fit various sized trapeziometacarpal joints. The implants 100, 200, 300 can be fabricated by folding or manipulating the allograft, xenograft or auto graft tissue onto itself or sectioning it to the appropriated size and machine sewn together with suture to create a compact block of tissue approximating the size of the trapezium.

Now that the implants according to various embodiments of the present invention have been described, methods of using the implants for surgical treatment of symptomatic trapezio metacarpal join arthritis will be discussed.

### Methods of Trapezio Metacarpal Joint Arthroplasty Using Implants

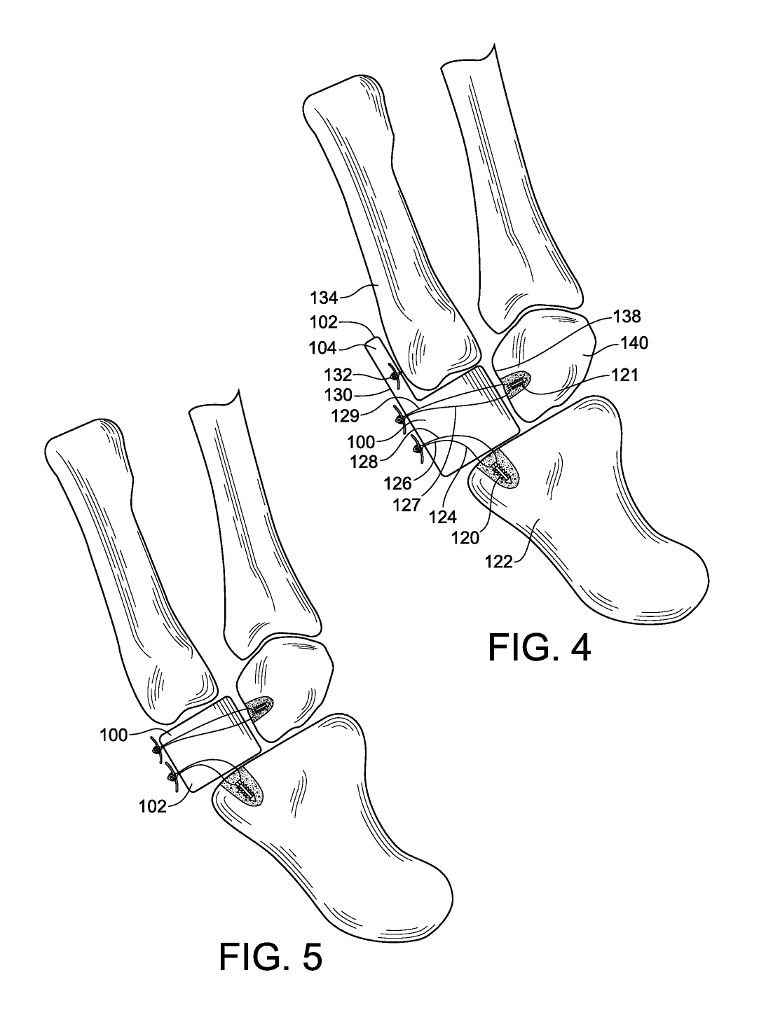

FIG. 4 shows the standard implant 100 used in a trapezio metacarpal joint arthroplasty method according to an embodiment of the present invention. In this embodiment, the trapezium is removed, such that the arthroplasty space is mobile and free of scar tissue. The arthroplasty space is measured and an appropriate right or left size implant is selected. Prior to placement of the implant 100, a small bone suture anchor 120 is placed into the center of the distal pole of the scaphoid 122. A suture 122, which is anchored at the scaphoid 122 by the bone suture anchor 120, has limbs 126, 128. Both limbs 126, 128 of the suture 124 are passed through a radial side 130 of the implant 100 at proximal third of the body 102 in a mattress fashion as shown in FIG. 4. In addition, a second small bone suture anchor 121 can be placed into the trapezoid radial face 138. The suture limbs 127, 129 are passed through the distal third of the implant in a mattress fashion as shown in FIG. 4. The implant 100 is then seated, and the limbs 126, 128, 127, 129 are tied together, thereby securing the implant 100 to the scaphoid 122 and the trapezoid 140. As discussed above, the radial extension 204 is optional. When the lateral subluxation of the thumb metacarpal is a concern, an implant including a radial extension is recommended. If lateral subluxation of the thumb metacarpal is not a concern, the radial extension 204 can be removed as shown in FIG. 5. In cases where lateral subluxation of the thumb metacarpal is a concern, the radial extension 204 is sutured using a suture 132 to the thumb radial metacarpal base 134 utilizing local soft tissue at the radial base of the thumb metacarpal. If there is inadequate soft tissue on the radial base of the thumb metacarpal, the radial extension can be secured the radial metacarpal base through bone tunnels or suture anchor(s) placed in the radial metacarpal base. If a volar approach is utilized, the thenar musculature and remaining capsule is repaired over the implant. If a dorsal approach is utilized, the capsule is approximated over the implant with non absorbable sutures.

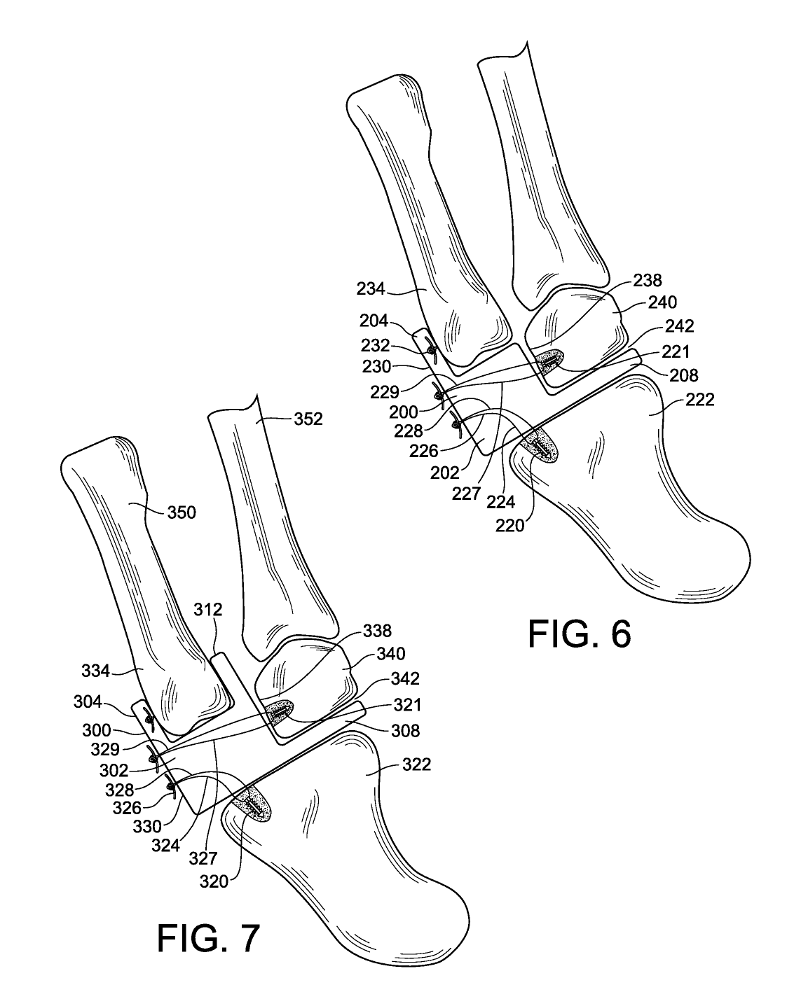

FIG. 6 shows the implant 200 used in a trapezio metacarpal joint arthroplasty method according to another embodiment of the present invention. In this embodiment, the trapezium is removed, such that the arthroplasty space is mobile and free of scar tissue. Further, about 2-3 mm of the proximal trapezoid 240 facing the scaphoid 222 is resected with an osteotome or small saw. The arthroplasty space is measured and an appropriate right or left size implant is selected. Prior to placement of the implant 200, a small bone suture anchor 220 is placed into the center of the distal pole of the scaphoid 222. Both limbs 226, 228 of a suture 224 are passed through the radial side 230 of the implant 200 at the proximal third of the body 202 in a mattress fashion. In addition, a second small bone suture anchor 221 can be placed into the trapezoid radial face 238. The suture limbs 227, 229 are passed through the distal third of the implant in a mattress fashion as shown in FIG. 6. The implant 200 is seated making sure the scaphotrapezoid extension 208 is seated into the scapho trapezoid arthroplasty space 242 that was created by resecting the proximal portion of the trapezoid 240. The limbs 226, 228, 227, 229 of the sutures, which are anchored at the suture anchors 220, 221 are then tied together to secure the implant 200 to the scaphoid 222 and to the trapezoid 240. As it was with the previous embodiment, the radial extension 204 is optional. If the lateral subluxation of the thumb metacarpal is a concern, then use of the radial extension 204 is recommended. If lateral subluxation of the thumb metacarpal is not a concern, the radial extension 204 can be resected. In cases where lateral subluxation of the thumb metacarpal is a concern, the radial extension 204 is sutured 232 to the thumb radial metacarpal base 234 utilizing local soft tissue at the radial base of the thumb metacarpal. If there is inadequate soft tissue on the radial base of the thumb metacarpal, the radial extension can be secured the radial metacarpal base through bone tunnels or suture anchor(s) placed in the radial metacarpal base. If a volar approach is utilized, the thenar musculature and remaining capsule is repaired over the implant. If a dorsal approach is utilized, the capsule is approximated over the implant with non absorbable sutures.

FIG. 7 shows the revision implant 300 used in a trapezio metacarpal joint arthroplasty method according to yet another embodiment of the present invention. In this embodiment, the trapezium is removed such that the arthroplasty space is mobile and free of scar tissue. When symptomatic scaphotrapezoid arthritis is present, about 2-3 millimeters of the proximal trapezoid 340 facing the scaphoid 322 is resected with an osteotome or small saw. If scapho trapezoid arthritis is not present, the scapho trapezoid joint is not addressed and the scaphotrapezoid extension 308 can be resected from the implant 300. The arthroplasty space is measured and an appropriate right or left size implant is selected. Prior to placement of the implant 300, a small bone suture anchor 320 is placed into the center of the distal pole of the scaphoid 322. In addition, a second small bone suture anchor 321 can be placed into the trapezoid radial face 338. The suture limbs 327, 329 are passed through the distal third of the implant in a mattress fashion as shown in FIG. 7. Both limbs 326, 328 of a suture 324 are passed through the radial side 330 of the revision implant 300 at the proximal third of the body 302 in a mattress fashion. If the scaphotrapezoid joint is addressed, the implant is seated making sure the scaphotrapezoid extension 308 is seated into the scapho trapezoid arthroplasty space 342 that was created by resecting the proximal portion of the trapezoid 340. The first/second metacarpal extension 312 is placed between the first metacarpal 352 and the second metacarpal 352 to provide a soft tissue interposition. Both limbs 326, 328 of the suture that is placed into the scaphoid 324 and both limbs 327, 329 of the suture anchor that is placed into the trapezoid 340 are then tied together to secure the implant to the scaphoid 322 and the trapezoid 340. The radial extension 304 is optional. If lateral subluxation of the thumb metacarpal is a concern, then use of the radial extension 304 is recommended. If lateral subluxation of the thumb metacarpal is not a concern, the radial extension 304 can be resected. In cases where lateral subluxation of the thumb metacarpal is a concern, the radial extension 304 is sutured to the thumb radial metacarpal base 334 utilizing local soft tissue at the radial base of the thumb metacarpal. If there is inadequate soft tissue on the radial base of the thumb metacarpal, the radial extension can be secured the radial metacarpal base through bone tunnels or suture anchor(s) placed in the radial metacarpal base. If a volar approach is utilized, the thenar musculature and remaining capsule is repaired over the implant. If a dorsal approach is utilized, the capsule is approximated over the implant with non absorbable sutures.

### Implants for Surgical Treatment of Symptomatic Arthritis of Scapho Trapezial Trapezoid (STT) Joint

An implant according to an embodiment of the present invention is designed as a soft tissue arthroplasty spacer that resurfaces the arthritic STT joint with a durable biologic material. It is also designed to stabilize the distal scaphoid to prevent volar flexion and carpal instability. It is designed to be simple to use, effective and time efficient. It obviates the need for autograft harvesting therefore reducing anesthesia and operating room time. It is more durable than auto graft tendon and will create a more durable resurfacing and arthroplasty spacer. The material properties of the implant are such that is easy to use and stable after suture fixation. Traditional tendon interposition arthroplasty can be difficult to reliably secure to bone and maintain position in the arthroplasty space without extrusion with loading of the joint.

This implant is designed as a durable biologic soft tissue spacer that resurfaces the arthritic STT joint resulting is significant symptomatic pain relief for patient with symptomatic STT arthritis that has not responded to conservative treatment. It is also designed to stabilize the distal pole of the scaphoid to prevent volar flexion and carpal instability.

FIG. 10 shows a Scapho Trapezial Trapezoid (STT) Biologic Arthroplasty Spacer according to an embodiment of the present invention. The STT Biologic Arthroplasty Spacer is also referred to herein as an implant, spacer, or other like terms. The STT implant 400 is made from human allograft, autograft or xenograft tissues including but not limited to tendon, dermal, fibro cartilage, labral or meniscal tissues. The implant 400 is designed as a biologic soft tissue spacer to resurface the distal pole of the scaphoid 422 and STT joint (FIG. 9). As shown in FIG. 10, the implant 400 includes a body 402 and a leg 404, which are either formed as a single piece or attached together with approximately 90° angle therebetween. Various sizes of the implant 400 can be made available to allow for complete resurfacing of the STT joint. The implant 400 can be fabricated by cutting folding, manipulating and or machine sewing allograft, xenograft or autograft into an appropriate shaped compact block of tissue that acts as arthroplasty spacer as well as resurfacing the STT joint.

### Methods of STT Joint Arthroplasty Using Implant

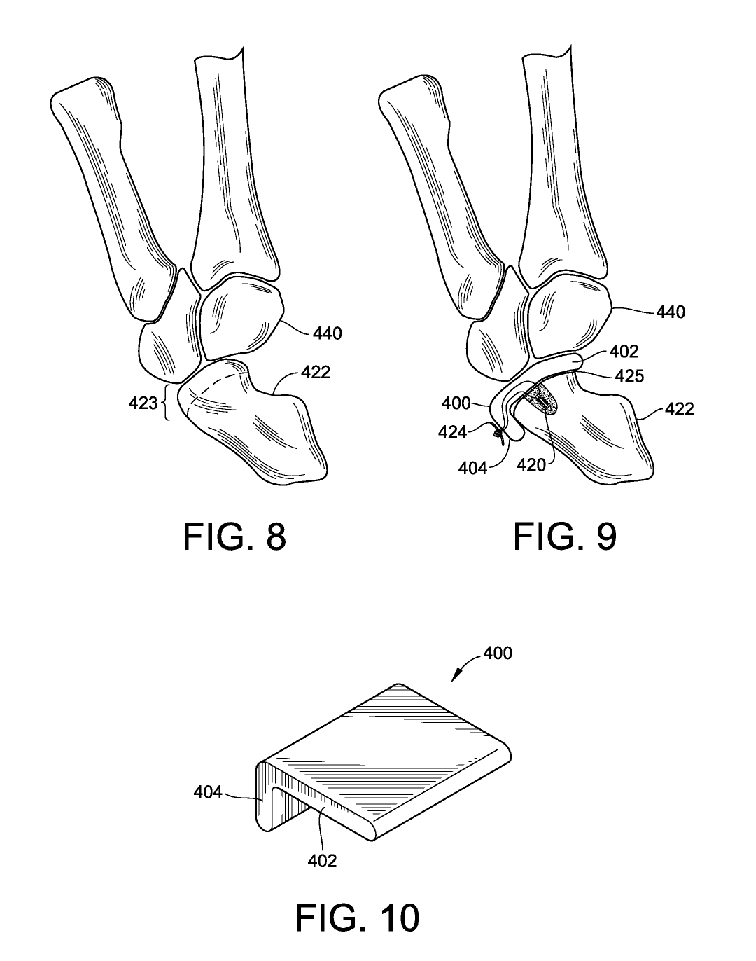

The implant 400 is designed to be placed through a volar or dorsoradial approach. According to one embodiment, approximately 2-3 mm 423 of distal scaphoid 422 is resected creating an arthroplasty space 425 as shown in FIGS. 8-9. A suture anchor 420 or a bone tunnel is placed into the distal scaphoid 422. The wrist is ulnarly deviated to facilitate placement of the implant. The implant 400 is seated in the STT joint making sure the entire distal scaphoid 422 is covered by the implant 400. The sutures 424, which are anchored to the scaphoid 422 via the suture anchore 420, are passed through the implant 400 exiting on the radial side. The sutures 424 are tied down securing the implant 400 to the scaphoid 422 and to prevent extrusion of the implant from the arthroplasty space. A non absorbable suture is then placed through the distal implant in the dorsal half of the implant in a mattress fashion. The suture is then passed through the dorsal STT joint capsule and/or through the overlying extensor carpi radialis longus tendon. The suture is not yet tied down. The wrist is slightly ulnarly deviated. Flouroscopic imaging is used to confirm the scaphoid is not volar flexed with a scapho lunate angle of less than 60 degrees. The scapholunate angle should be 60 degrees or less. Using a power drill, a smooth 0.045″, 0.055″ or 0.065″ K-wire is then placed through the distal third of the scaphoid in a radial to ulnar direction and driven into the capitate stabilizing the scaphoid. The previously placed suture is now tied down stabilizing the distal pole of the scaphoid to the dorsal capsule and/or overlying extensor carpi radialis brevis tendon to prevent volar flexion of the scaphoid.

### After Care

The patient is placed into a thumb spica forearm based splint with the wrist slightly ulnarly deviated. At eight weeks status post surgery the “K” wire is removed and the wrist is mobilized. Strengthening is begun at ten weeks s/p surgery.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An arthroplasty spacer, comprising:
a body configured to fit in an arthroplasty space; and
wherein the arthroplasty spacer is formed of a biologic soft tissue.

2. The arthroplasty spacer of claim 1, wherein the body is configured to replace a resected trapezium for a trapezio metacarpal joint arthroplasty, wherein the body is slightly larger than the resected trapezium.

3. The arthroplasty spacer of claim 2, wherein the body has a rectangular box like shape; and further including a radial extension extending generally vertically upward from a first side of the body; wherein the body is placed in the arthroplasty space created from the resected trapezium, and the radial extension is placed adjacent a thumb radial metacarpal base to prevent lateral subluxation of the thumb metacarpal.

4. The arthroplasty spacer of claim 3, further including a scaphotrapezoid extension extending generally horizontally from a second side of the body, the first side and the second side being on opposite ends of the body, such that the radial extension, the body, and the scaphotrapezoid extension form a stair-like shape; wherein the scaphotrapezoid extension is configured to cover an approximate area of a scapho trapezoid joint and interposed into the scapho trapezoid joint in an arthroplasty space created from a partial resection of a trapezoid to resurface an arthritic scapho trapezoid joint.

5. The arthroplasty spacer of claim 4, further including a first/second metacarpal extension extending generally vertically upward from the second side of the body, such that the first/second metacarpal extension and the second side of the body and the scaphotrapezoid extension generally form L-like shape, and the radial extension and a third side of the body and the first/second metacarpal extension generally form a U-like shape; wherein the first/second metacarpal extension is place between first and second metacarpals to provide a soft tissue interposition.

6. The arthroplasty spacer of claim 1, wherein the body is configured to fit the arthroplasty space created from a partial resection of a distal scaphoid to resurface an arthritic scapho trapezial trapezoid (STT) joint.

7. The arthroplasty spacer of claim 1, wherein the biologic soft tissue is human allograft, autograft or xenograft tissues.

8. The arthroplasty spacer of claim 7, wherein the biologic soft tissue is selected from a group consisting of tendon, dermal, fibro cartilage, labral, meniscal tissues and a mixture thereof.

9. The arthroplasty spacer of claim 1, wherein the body is a compact block of biologic soft tissue formed by folding or manipulating the biologic soft tissue onto itself or sectioning, which is sewn together using a suture.

10. A method for surgically treating symptomatic trapezio metacarpal joint arthritis, comprising:
removing a trapezium;
preparing an arthroplasty space created from the removed trapezium, such that the arthroplasty space is mobile and free of scar tissue;
measuring a size of the arthroplasty space;
selecting a biologic soft tissue spacer according to the size of the arthroplasty space;
placing the biologic soft tissue spacer in the arthroplasty space; and
securing the biologic soft tissue spacer.

11. The method of claim 10, wherein the biologic soft tissue spacer is formed of human allograft, autograft or xenograft tissues and configured to replace the trapezium; wherein the biologic soft tissue spacer includes a body, the body configured to be slightly larger than the trapezium.

12. The method of claim 11, wherein the biologic soft tissue spacer further includes a radial extension; wherein placing the biologic soft tissue spacer includes arranging the body in the arthroplasty space created from the removed trapezium and placing the radial extension adjacent a thumb radial metacarpal base to prevent lateral subluxation of the thumb metacarpal.

13. The method of claim 12, further including securing the radial extension to the thumb radial metacarpal base by suturing to local soft tissue at the thumb radial metacarpal base or via a bone tunnel or a suture anchor placed in the thumb radial metacarpal base.

14. The method of claim 11, further including resecting about 2-3 mm of a proximal trapezoid, and preparing a scapho trapezoid arthroplasty space created therein; wherein the biologic soft tissue spacer further includes a scaphotrapezoid extension extending from the body; and wherein placing the biologic soft tissue spacer includes arranging the body in the arthroplasty space created from the removed trapezium and placing the scaphotrapezoid extension in the scapho trapezoid arthroplasty space to also treat a symptomatic scapho trapezial arthritis.

15. The method of claim 11, wherein the biologic soft tissue spacer further includes a first/second metacarpal extension, wherein placing the biologic soft tissue spacer includes arranging the body in the arthroplasty space created from the removed trapezium and placing the first/second metacarpal extension between a first metacarpal and a second metacarpal to provide a soft tissue interposition to treat a symptomatic first/second metacarpal arthritis or impingement.

16. The method of claim 10, wherein securing the biologic soft tissue spacer includes placing a suture anchor into around the center of a distal pole of the scaphoid, securing a suture having limbs to the suture anchor, passing the limbs through the biologic soft tissue spacer, and tying the limbs together on a radial side of the biologic soft tissue spacer, such that the biologic soft tissue spacer is secured to the scaphoid.

17. The method of claim 16, wherein securing the biologic soft tissue spacer further includes placing a second suture anchor into a trapezoid radial face, securing a second suture having limbs to the second suture anchor, passing the limbs through the biologic soft tissue spacer, and tying the limbs together on the radial side of the biologic soft tissue spacer to secure the biologic soft tissue spacer to the trapezoid.

18. The method of claim 10, further including repairing a thenar musculature and a remaining capsule over the biologic soft tissue spacer when a volar approach is used.

19. The method of claim 10, further including repairing capsule over the biologic tissue spacer using non-absorbable sutures when a dorsal approach is used.

20. A method for surgically treating symptomatic arthritis of scapho trapezial trapezoid (STT) joint, comprising:
resecting about 2-3 mm of a distal scaphoid to create an arthroplasty space;
measuring a surface area of the distal scaphoid in the STT joint;
selecting a biologic soft tissue spacer according to the surface area of the distal scaphoid;
placing the biologic soft tissue spacer in the arthroplasty space; and
securing the biologic soft tissue spacer to the distal scaphoid.

21. The method of claim 20, wherein placing the biologic soft tissue spacer includes deviating a wrist ulnarly and setting the biologic soft tissue spacer in the STT joint such that the biologic soft tissue spacer generally covers the entire distal scaphoid; wherein securing the biologic soft tissue spacer includes placing a suture anchor into the distal scaphoid before setting the biologic soft tissue spacer in the STT joint, and passing limbs of a suture anchor suture through the biologic soft tissue spacer, and tying the limbs together on a radial side of the biologic soft tissue spacer, such that the biologic soft tissue spacer is secured to the scaphoid.

22. The method of claim 20, further including placing a non-absorbable suture through the dorsal half of the biologic soft tissue spacer in a mattress fashion, and passing the suture through a dorsal STT joint capsule and through a overlaying extensor carpi radialis longus tendon; confirming that a scapholunate angle is 60° or less using a flouroscopic imaging; placing a smooth 0.045″, 0.055″ or 0.065″ K-wire through a distal third of the scapohid in a radial to ulnar direction and driving the K-wire into a capitate using a power drill to stabilize the scaphoid; and tying the suture to stabilize a distal pole of the scaphoid to the dorsal capsule and the overlaying extensor carpi radialis brevis tendon to prevent volar flexion of the scaphoid.
